(19) United States
(12) Patent Application Publication
(10) Pub. No.: US 2004/0247549 A1
(43) Pub. Date: Dec. 9, 2004

(54) Cosmetic emulsions containing at least one hetero polymer and at least one sunscreen and methods of using the same

(75) Inventors: Shao Xiang Lu, Plainsboro, NJ (US); Mohamed Kanji, Edison, NJ (US)

Correspondence Address: LERNER, DAVID, LITTENBERG,, KRUMHOLZ & MENTLIK, 600 SOUTH AVENUE WEST, WESTFIELD, NJ, 07090, US

(73) Assignee: L'OREAL S.A., Paris (FR)

(21) Appl. No.: 10/747,412

(22) Filed: Dec. 22, 2003

Related U.S. Application Data
(63) Continuation-in-part of application No. 10/618,315, filed on Jul. 11, 2003.
(63) Continuation-in-part of application No. 10/459,636, filed on Jun. 12, 2003.
(60) Provisional application No. 60/387,602, filed on Jun. 12, 2002.

(51) Int. Cl. A61K 7/06; A61K 7/11

(52) U.S. Cl. 424/70.12

(57) ABSTRACT

The present invention provides, in one embodiment, a transfer resistant cosmetic composition, which may also be pliable and/or comfortable to wear upon application to a substrate, and which also exhibit unexpected increases in Sun Protection Factor, and contain at least one structuring polymer, at least one at least one sunscreen agent, a silicone elastomer powder comprising a silicone elastomer core coated with a silicone resin, and a swelling agent for the powder. These compositions may be used cosmetically, dermatologically, and pharmaceutically.

### CROSS-REFERENCE TO RELATED APPLICATIONS

This application is a CIP of U.S. application Ser. No. 10/618,315, filed Jul. 11, 2003, and U.S. application Ser. No. 10/459,636, filed Jun. 12, 2003, which claims benefit of U.S. Provisional Application No. 60/387,602, filed Jun. 12, 2002, the contents of all of which are hereby incorporated by reference.

### BACKGROUND OF THE INVENTION

Many cosmetic compositions, including pigmented cosmetics such as foundations, concealers, lipsticks, mascaras, and other cosmetic and sunscreen compositions, have been developed for longer wear and non-transfer properties. This is accomplished by the use of compositions that form a film after application. Such compositions generally contain volatile solvents that evaporate on contact with the skin or other keratinous tissue, leaving behind a layer comprising waxes and/or resins, pigments, fillers, and actives. However, these compositions tend to be uncomfortable for the wearer as the composition remains on the skin or other keratinous tissue as a brittle or non-flexible film. Such compositions may not be either pliable or soft, and they may not be comfortable to wear. There may also be a tendency for such compositions to flake off because of poor adherence to the skin or other keratinous tissue. Furthermore, compositions may have a tendency to be tacky, resulting in poor application and spreadability characteristics. Furthermore, the addition of sunscreens does not always result in the SPF (Sun Protection Factor) value being attained with a known concentration of sunscreen agent.

The need therefore still remains for improved long-wearing cosmetic compositions which transfer little or not at all, i.e., “transfer-free” or transfer resistant compositions which also possess good cosmetic properties such as pliability and comfort and which provide good sun protection. For example, a composition that is transfer-resistant may deposit a film onto a keratinous substance that may not transfer when the keratinous substance comes into contact with, for example, skin, a cup, paper, cigarette, or a handkerchief.

### SUMMARY OF THE INVENTION

To achieve at least one of these and other advantages, the present invention, in one aspect, provides a composition comprising at least one structuring polymer comprising a polymer skeleton which comprises at least one hydrocarbon-based repeating unit comprising at least one hetero atom, at least one sunscreen agent, a silicone elastomer powder comprising a silicone elastomer core coated with a silicone resin, and a swelling agent for the powder. In some embodiments, the polymer skeleton contains at least one amide repeating unit. It has been unexpectedly found that the addition of the structuring polymer increases the SPF value of the composition.

The invention, in another aspect, provides compositions comprising at least one structuring polymer, at least one sunscreen, a film-forming polymer, a silicone elastomer powder comprising a silicone elastomer core coated with a silicone resin; and a swelling agent for the powder. In one embodiment, the at least one structuring polymer and the film-forming polymer are present in an amount effective to provide transfer resistant properties, and may also provide at least one of the following properties: pliability, softness, and wearing comfort.

Yet another aspect of the present invention is directed to a foundation, mascara, eye liner, concealer, lipstick, blush for cheeks or eyelids, body makeup, sun screen, deodorant, colorant for skin or hair, skin care formula, shampoo, after shampoo treatment, or makeup removing product comprising:

at least one liquid fatty phase in said foundation, mascara, eye liner, concealer, lipstick, blush for cheeks or eyelids, body makeup, sun screen, deodorant, colorant for skin or hair, skin care formula, shampoo, after shampoo treatment, or makeup removing product which comprises:

at least one structuring polymer comprising:

a polymer skeleton which comprises at least one hydrocarbon-based repeating unit comprising at least one hetero atom;

(ii) at least one sunscreen agent; and

(iii) a silicone elastomer powder comprising a silicone elastomer core coated with a silicone resin; and a swelling agent for the powder.

A further aspect of the present invention is directed to a make-up and/or care and/or treatment composition for keratinous fibers comprising:

at least one liquid fatty phase in said composition which comprises:

at least one structuring polymer comprising:

a polymer skeleton which comprises at least one hydrocarbon-based repeating unit comprising at least one hetero atom;

(ii) at least one sunscreen agent;

(iii) a silicone elastomer powder comprising a silicone elastomer core coated with a silicone resin; and

(iv) a swelling agent for the powder.

A further aspect of the present invention is directed to a treatment, care or make-up composition for keratinous fibers comprising a structured composition containing at least one liquid fatty phase in the treatment, care or make-up composition structured with at least one structuring polymer comprising a polymer skeleton comprising at least one hydrocarbon-based repeating unit comprising at least one hetero atom, at least one sunscreen agent, a silicone elastomer powder comprising a silicone elastomer core coated with a silicone resin, a swelling agent for the powder, and at least one coloring agent.

A further aspect of the present invention is directed to a method for care, make up, or treatment of a keratin material chosen from lips, skin, and keratinous fibers, comprising the application to said keratin material of a cosmetic composition comprising:

at least one liquid fatty phase which comprises:

at least one structuring polymer comprising:

a polymer skeleton which comprises at least one hydrocarbon-based repeating unit comprising at least one hetero atom;

at least one sunscreen agent;

a silicone elastomer powder comprising a silicone elastomer core coated with a silicone resin; and

a swelling agent for the powder.

A further aspect of the present invention is directed to a method for making a cosmetic composition in the form of a physiologically acceptable composition comprising including in said composition

at least one liquid fatty phase which comprises:

at least one structuring polymer comprising a polymer skeleton which comprises at least one hydrocarbon-based repeating unit comprising at least one hetero atom;

at least one sunscreen agent;

a silicone elastomer powder comprising a silicone elastomer core coated with a silicone resin; and

a swelling agent for the powder.

It is to be understood that both the foregoing general description and the following detailed description are exemplary and explanatory only, and are not restrictive of the invention.

### DETAILED DESCRIPTION

As defined herein, the sun protection factor (SPF) is expressed mathematically by the ratio of the irradiation time required to attain the erythema-forming threshold with the UV screening agent to the time required to attain the erythema-forming threshold in the absence of UV screening agent. Measurement of the levels of sunscreen protection or SPF is performed according to the test set forth in the Federal Register Vol. 46, No. 17, of Tuesday Jan. 27, 1981.

As defined herein, to test stability, the composition is placed in a controlled environment chamber for 8 weeks at 25° C. In this test, the physical condition of the sample is inspected as it is placed in the chamber. The sample is then inspected again at 24 hours, 3 days, 1 week, 2 weeks, 4 weeks and 8 weeks. At each inspection, the sample is examined for abnormalities in the composition such as bending or leaning if the composition is in stick form, phase separation, melting, or syneresis. As used herein, syneresis is the appearance of droplets on the surface of a composition that are visible to the naked eye. The stability is further tested by repeating the 8-week test at 4° C., 37° C., 45° C., and 50° C., and under freeze-thaw conditions. A composition is considered to lack stability if in any of these tests an abnormality that impedes functioning of the composition is observed. The skilled artisan will readily recognize an abnormality that impedes functioning of a composition based on the intended application.

Structured liquid fatty phases in cosmetic or dermatological products are known in the art. As used herein, “structured” means gelled and/or rigidified. Structured liquid fatty phases may be found in solid compositions such as deodorants, balms, lip compositions, concealer products and cast foundations.

The compositions of the invention, in one embodiment, may comprise at least one liquid fatty phase. As used herein, “liquid fatty phase” means a fatty phase which is liquid at room temperature (25° C.) and at atmospheric pressure (760 mmHg) and which is composed of at least one fatty substance, such as an oil, which is liquid at room temperature and not soluble in water. If the at least one liquid fatty phase comprises two or more fatty substances, they should be mutually compatible.

Structured liquid fatty phases may make it possible to control the exudation of the liquid fatty phase from the solid compositions of which they are components including exudation in a wet or hot atmosphere or environment. Structuring of the liquid fatty phase may also limit bleeding of this phase outside of the intended area of application and especially into wrinkles and fine lines after it has been deposited, for example, on keratinous material. As used herein, “keratinous material” is meant to comprise hair, lips, skin, scalp and superficial body growths such as eyelashes, eyebrows and nails.

The invention applies not only to make-up products for at least one keratinous material such as lip compositions, lip pencils, foundations including foundations which may be cast in the form of a stick or a dish, concealer products, temporary tattoo products, eyeliners, mascara bars but also to body hygiene products such as deodorant sticks, and to care products and products for treating at least one keratinous material such as sunscreen and after-sun products which may be in stick form. The present invention may be in the form of mascara product including mascara bars, an eyeliner product, a foundation product, a lipstick product, a blush for cheeks or eyelids, a deodorant product, a make-up product for the body, a make-up-removing product, an eyeshadow product, a face powder product, a concealer product, a treating shampoo product, a hair conditioning product, a sun screen, colorant for the skin or hair, or skin care formula such as, for example, anti-pimple or shaving cut formulas. As defined herein, a deodorant product is a body hygiene product and does not relate to care, make-up, or treatment of keratin materials, including keratin fibers, skin, or lips.

For example, the composition of the present invention may be in a form chosen from a paste, a solid, a gel, and a cream. It may be an emulsion, such as an oil-in-water or water-in-oil emulsion, a multiple emulsion, such as an oil-in-water-in-oil emulsion or a water-in-oil-in-water emulsion, or a solid, rigid or supple gel, including anhydrous gels. In one embodiment, the composition of the invention is anhydrous. The composition of the invention may, for example, comprise an external or continuous fatty phase. In another embodiment, the composition of the invention is transparent or clear, including for example, a composition without pigments. The composition can also be in a form chosen from a translucent anhydrous gel and a transparent anhydrous gel. The composition can also be a molded composition or cast as a stick or a dish. The composition in one embodiment is a solid such as a molded stick or a poured stick.

In one embodiment, the at least one structuring polymer in the composition of the invention is a solid that is not deformable at room temperature (25° C.) and atmospheric pressure (760 mmHg). In a further embodiment, the at least one structuring polymer is capable of structuring the composition without opacifying it. As defined above, the at least one structuring polymer of the present invention comprises a polymer skeleton comprising at least one hydrocarbon-based repeating unit comprising at least one hetero atom. In one embodiment, the at least one structuring polymer further comprises at least one terminal fatty chain chosen from alkyl and alkenyl chains, such as of at least 4 atoms, and further such as comprising 8 to 120 carbon atoms, bonded to the polymer skeleton via at least one linking group. The terminal fatty chain may, for example, be functionalized. The at least one structuring polymer may also further comprise at least one pendant fatty chain chosen from alkyl and alkenyl chains, such as of at least 4 atoms, and further such as comprising 8 to 120 carbon atoms, bonded to any carbon or hetero atom of the polymer skeleton via at least one linking group. The pendant fatty chain may, for example, be functionalized. The at least one structuring polymer may comprise both at least one pendant fatty chain and at least one terminal fatty chain as defined above, and one or both types of chains can be functionalized.

In one embodiment, the structuring polymer comprises at least two hydrocarbon-based repeating units. As a further example, the structuring polymer comprises at least three hydrocarbon-based repeating units and as an even further example, the at least three repeating units are identical.

As used herein, “functionalized” means comprising at least one functional group. Non-limiting examples of functional groups include hydroxyl groups, ether groups, oxyalkylene groups, polyoxyalkylene groups, carboxylic acid groups, amine groups, amide groups, halogen containing groups, including fluoro and perfluoro groups, halogen atoms, ester groups, siloxane groups and polysiloxane groups.

For purposes of the invention, the expression “functionalized chain” means, for example, an alkyl chain comprising at least one functional (reactive) group chosen, for example, from those recited above. For example, in one embodiment, the hydrogen atoms of at least one alkyl chain may be substituted at least partially with fluorine atoms.

According to the invention, these chains may be linked directly to the polymer skeleton or via an ester function or a perfluoro group.

For the purposes of the invention, the term “polymer” means a compound containing at least 2 repeating units, such as, for example, a compound containing at least 3 repeating units, which may be identical.

As used herein, the expression “hydrocarbon-based repeating unit” includes a repeating unit comprising from 2 to 80 carbon atoms, such as, for example, from 2 to 60 carbon atoms. The at least one hydrocarbon-based repeating unit may also comprise oxygen atoms. The hydrocarbon-based repeating unit may be chosen from saturated and unsaturated hydrocarbon-based repeating units which in turn may be chosen from linear hydrocarbon-based repeating units, branched hydrocarbon-based repeating units and cyclic hydrocarbon-based repeating units. The at least one hydrocarbon-based repeating unit may comprise, for example, at least one hetero atom that is part of the polymer skeleton, i.e., not pendant. The at least one hetero atom may be chosen, for example, from nitrogen, sulphur, and phosphorus. For example, the at least one hetero atom may be a nitrogen atom, such as a non-pendant nitrogen atom. In another embodiment, the at least one hydrocarbon-based repeating unit may comprise at least one hetero atom with the proviso that the at least one hetero atom is not nitrogen. In another embodiment, the at least one hetero atom is combined with at least one atom chosen from oxygen and carbon to form a hetero atom group. In one embodiment, the hetero atom group comprises a carbonyl group.

The at least one repeating unit comprising at least one hetero atom may be chosen, for example, from amide groups, carbamate groups, and urea groups. In one embodiment, the at least one repeating unit comprises amide groups forming a polyamide skeleton. In another embodiment, the at least one repeating unit comprises carbamate groups and/or urea groups forming a polyurethane skeleton, a polyurea skeleton and/or a polyurethane-polyurea skeleton. The pendant chains, for example, can be linked directly to at least one of the hetero atoms of the polymer skeleton. In yet another embodiment, the at least one hydrocarbon-based repeating unit may comprise at least one hetero atom group with the proviso that the at least one hetero atom group is not an amide group. In one embodiment, the polymer skeleton comprises at least one repeating unit chosen from silicone units and oxyalkylene units, the at least one repeating unit being between the hydrocarbon-based repeating units.

In one embodiment, the compositions of the invention comprise at least one structuring polymer with nitrogen atoms, such as amide, urea, or carbamate units, such as amide units, and at least one polar oil.

In one embodiment, in the at least one structuring polymer, the percentage of the total number of fatty chains ranges from 40% to 98% relative to the total number of repeating units and fatty chains, and as a further example, from 50% to 95%. In a further embodiment wherein the polymer skeleton is a polyamide skeleton, in the at least one structuring polymer, the percentage of the total number of fatty chains ranges from 40% to 98% relative to the total number of all amide units and fatty chains, and as a further example, from 50% to 95%.

In a further embodiment, the nature and proportion of the at least one hydrocarbon-based repeating unit comprising at least one hetero atom depends on the nature of a liquid fatty phase of the composition and is, for example, similar to the nature of the fatty phase. For example, not to be limited as to theory, the more polar the hydrocarbon-based repeating units containing a hetero atom, and in high proportion, which corresponds to the presence of several hetero atoms, the greater the affinity of the at least one structuring polymer to polar oils. Conversely, the more non-polar, or even apolar, and lesser in proportion the hydrocarbon-based repeating units containing a hetero atom, the greater the affinity of the polymer for apolar oils.

In another embodiment, the invention is drawn to a structured composition containing at least one liquid fatty phase structured with at least one structuring polymer, wherein the at least one structuring polymer is a polyamide comprising a polymer skeleton comprising at least one amide repeating unit and optionally at least one pendant fatty chain and/or at least one terminal chain that are optionally functionalized and comprise from 8 to 120 carbon atoms, bonded to at least one of the amide repeating units via at least one linking group. The liquid fatty phase further contains at least one organogellator for gelling the liquid fatty phase. The at least one liquid fatty phase, the at least one structuring polyamide and the at least one organogellator together form a physiologically acceptable medium.

When the structuring polymer has amide repeating units, the pendant fatty chains may be linked to at least one of the nitrogen atoms in the amide repeating units.

The structuring polymer, for example the polyamide polymer, may have a weight-average molecular mass of less than 100,000, such as less than 50,000. In another embodiment, the weight-average molecular mass may range from 1000 to 30,000, such as from 2000 to 20,000, further such as from 2000 to 10,000.

As discussed, the at least one structuring polymer may, for example, be chosen from polyamide polymers. A polyamide polymer may comprise, for example, a polymer skeleton that comprises at least one amide repeating unit, i.e., a polyamide skeleton. In one embodiment, the polyamide skeleton may further comprise at least one terminal fatty chain chosen from alkyl chains, for example, alkyl chains comprising at least four carbon atoms, and alkenyl chains, for example, alkenyl chains comprising at least four carbon atoms, bonded to the at least one polyamide skeleton via at least one linking group, and/or at least one pendant fatty chain chosen from alkyl chains, for example, alkyl chains comprising at least four carbon atoms, and alkenyl chains, for example, alkenyl chains comprising at least four carbon atoms, bonded to the at least one polyamide skeleton via at least one linking group. In one embodiment, the polyamide skeleton may comprise at least one terminal fatty chain chosen from fatty chains comprising 8 to 120 carbon atoms, such as, for example, 12 to 68 carbon atoms, bonded to the at least one polyamide skeleton via at least one linking group and/or at least one pendant fatty chain chosen from fatty chains comprising 8 to 120 carbon atoms, such as, for example, 12 to 68 carbon atoms, bonded to the at least one polyamide skeleton via at least one linking group, such as bonded to any carbon or nitrogen of the polyamide skeleton via the at least one linking group. In one embodiment, the at least one linking group is chosen from single bonds and urea, urethane, thiourea, thiourethane, thioether, thioester, ester, ether and amine groups. For example, the at least one linking group is chosen from ureas, esters, and amines, and as a further example, is chosen from esters and amines. The bond is, for example, an ester bond. In one embodiment, these polymers comprise a fatty chain at each end of the polymer skeleton, such as the polyamide skeleton.

In one embodiment, due to the presence of at least one chain, the polyamide polymers may be readily soluble in oils (i.e., water-immiscible liquid compounds) and thus may give macroscopically homogeneous compositions even with a high content (at least 25%) of the polyamide polymers, unlike certain polymers of the prior art that do not contain such alkyl or alkenyl chains at the end of the polyamide skeleton. As defined herein, a composition is soluble if it has a solubility of greater than 0.01 g per 100 ml of solution at 250° C.

In a further embodiment, the polyamide polymers can be chosen from polymers resulting from at least one polycondensation reaction between at least one acid chosen from dicarboxylic acids comprising at least 32 carbon atoms, such as 32 to 44 carbon atoms, and at least one amine chosen from diamines comprising at least 2 carbon atoms, such as from 2 to 36 carbon atoms, and triamines comprising at least 2 carbon atoms, such as from 2 to 36 carbon atoms. The dicarboxylic acids can, for example, be chosen from dimers of at least one fatty acid comprising at least 16 carbon atoms, such as oleic acid, linoleic acid and linolenic acid. The at least one amine can, for example, be chosen from diamines, such as ethylenediamine, hexylenediamine, hexamethylenediamine, phenylenediamine and triamines, such as ethylenetriamine.

The polyamide polymers may also be chosen from polymers comprising at least one terminal carboxylic acid group. The at least one terminal carboxylic acid group can, for example, be esterified with at least one alcohol chosen from monoalcohols comprising at least 4 carbon atoms. For example, the at least one alcohol can be chosen from monoalcohols comprising from 10 to 36 carbon atoms. In a further embodiment, the monoalcohols can comprise from 12 to 24 carbon atoms, such as from 16 to 24 carbon atoms, and for example 18 carbon atoms.

In one embodiment, the at least one polyamide polymer may be chosen from those described in U.S. Pat. No. 5,783,657, the disclosure of which is incorporated herein by reference, which are polymers of formula (I):

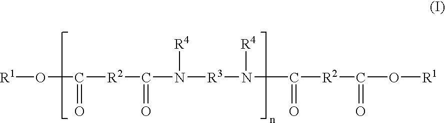

in which:

n is an integer which represents the number of amide units such that the number of ester groups present in said at least one polyamide polymer ranges from 10% to 50% of the total number of all said ester groups and all said amide groups comprised in said at least one polyamide polymer;

R¹, which are identical or different, are each chosen from alkyl groups comprising at least 4 carbon atoms and alkenyl groups comprising at least 4 carbon atoms. In one embodiment, the alkyl group comprises from 4 to 24 carbon atoms and the alkenyl group comprises from 4 to 24 carbon atoms;

R², which are identical or different, are each chosen from C₄ to C₄₂ hydrocarbon-based groups with the proviso that at least 50% of all R² are chosen from C₃₀ to C₄₂ hydrocarbon-based groups;

R³, which are identical or different, are each chosen from organic groups comprising atoms chosen from carbon atoms, hydrogen atoms, oxygen atoms and nitrogen atoms with the proviso that R³ comprises at least 2 carbon atoms; and

R⁴, which are identical or different, are each chosen from hydrogen atoms, C₁ to C₁₀ alkyl groups and a direct bond to at least one group chosen from R³ and another R⁴ such that when said at least one group is chosen from another R⁴, the nitrogen atom to which both R³ and R⁴ are bonded forms part of a heterocyclic structure defined in part by R⁴—N—R³, with the proviso that at least 50% of all R⁴ are chosen from hydrogen atoms.

In one embodiment, the at least one terminal fatty chain of formula (I) is linked to the last hetero atom, in this case nitrogen, of the polyamide skeleton. In a further embodiment, the terminal chains are functionalized. In another embodiment, the ester groups of formula (I), are linked to the terminal and/or pendant fatty chains, represent from 15% to 40% of the total number of ester and amide groups, such as, for example, from 20% to 35%.

In one embodiment, n may be an integer ranging from 1 to 5, for example, an integer ranging from 3 to 5. In the present invention, R¹, which are identical or different, can, for example, each be chosen from C₁₂ to C₂₂ alkyl groups, such as from C₁₆ to C₂₂ alkyl groups.

In the present invention, R², which are identical or different, can, for example, each be chosen from C₁₀ to C₄₂ alkyl groups. At least 50% of all R², which are identical or different, can, for example, each be chosen from groups comprising from 30 to 42 carbon atoms. At least 75% of all R², which are identical or different, can, for example, each be chosen from groups comprising from 30 to 42 carbon atoms. In the two aforementioned embodiments, the remaining R², which are identical or different, can, for example, each be chosen from C₄ to C₁₉ groups, such as C₄ to C₁₂ groups.

R³, which can be identical or different, can, for example, each be chosen from C₂ to C₃₆ hydrocarbon-based groups and polyoxyalkylene groups. In another example, R³, which can be identical or different, can each for example, be chosen from C₂ to C₁₂ hydrocarbon-based groups. In another embodiment, R⁴, which can be identical or different, can each be chosen from hydrogen atoms. As used herein, hydrocarbon-based groups may be chosen from linear, cyclic and branched, and saturated and unsaturated groups. The hydrocarbon-based groups can be chosen from aliphatic and aromatic groups. In one example, the hydrocarbon-based groups are chosen from aliphatic groups. The alkyl and alkylene groups may be chosen from linear, cyclic and branched, and saturated and unsaturated groups.

In general, the pendant and terminal fatty chains may be chosen from linear, cyclic and branched, and saturated and unsaturated groups. The pendant and terminal fatty chains can be chosen from aliphatic and aromatic groups. In one example, the pendant and terminal fatty chains are chosen from aliphatic groups.

According to the invention, the structuring of the liquid fatty phase is obtained with the aid of at least one structuring polymer, such as the at least one polymer of formula (I). The at least one polyamide polymer of formula (I) may, for example, be in the form of a mixture of polymers, and this mixture may also comprise a compound of formula (I) wherein n is equal to zero, i.e., a diester.

Non-limiting examples of at least one polyamide polymer that may be used in the composition according to the present invention include the commercial products sold by Arizona Chemical under the names Uniclear 80 and Uniclear 100. These are sold, respectively, in the form of an 80% (in terms of active material) gel in a mineral oil and a 100% (in terms of active material) gel. These polymers have a softening point ranging from 88° C. to 94° C., and may be mixtures of copolymers derived from monomers of (i) C₃₆ diacids and (ii) ethylenediamine, and have a weight-average molecular mass of about 6000. Terminal ester groups result from esterification of the remaining acid end groups with at least one alcohol chosen from cetyl alcohol and stearyl alcohol. A mixture of cetyl and stearyl alcohols is sometimes called cetylstearyl alcohol.

Other non-limiting examples of at least one polyamide polymer that may be used in the composition according to the present invention include polyamide polymers resulting from the condensation of at least one aliphatic dicarboxylic acid and at least one diamine, the carbonyl and amine groups being condensed via an amide bond. Examples of these polyamide polymers are those sold under the brand name Versamid by the companies General Mills Inc. and Henkel Corp. (Versamid 930, 744 or 1655) or by the company Olin Mathieson Chemical Corp. under the brand name Onamid, in particular Onamid S or C. These resins have a weight-average molecular mass ranging from 6000 to 9000. For further information regarding these polyamides, reference may be made to U.S. Pat. Nos. 3,645,705 and 3,148,125, the disclosures of which are hereby incorporated by reference.

Other examples of polyamides include those sold by the company Arizona Chemical under the references Uni-Rez (2658, 2931, 2970, 2621, 2613, 2624, 2665, 1554, 2623 and 2662) and the product sold under the reference Macromelt 6212 by the company Henkel. For further information regarding these polyamides, reference may be made to U.S. Pat. No. 5,500,209, the disclosure of which is hereby incorporated by reference. Such polyamides display high melt viscosity characteristics. MACROMELT 6212, for example, has a high melt viscosity at 190° C. of 30-40 poise (as measured by a Brookfield Viscometer, Model RVF #3 spindle, 20 RPM).

In a further embodiment, the at least one polyamide polymer may be chosen from polyamide resins from vegetable sources. Polyamide resins from vegetable sources may be chosen from, for example, the polyamide resins of U.S. Pat. Nos. 5,783,657 and 5,998,570, the disclosures of which are herein incorporated by reference.

In one embodiment, the at least one polyamide polymer may be present in the composition in an amount ranging, for example, from 0.5% to 80%, such as from 2% to 60%, further such as from 5% to 40%, by weight relative to the total weight of the composition. In a further embodiment the at least one polyamide polymer may be present in the composition in an amount ranging, for example, from 5% to 25% by weight relative to the total weight of the composition.

In one embodiment, the at least one structuring polymer in the composition according to the invention corresponds to the polyamide polymers of formula (I). Due to fatty chain(s), these polymers may be readily soluble in oils and thus lead to compositions that are macroscopically homogeneous even with a high content (at least 25%) of at least one structuring polymer.

The at least one structuring polymer may have a softening point greater than 50° C., such as from 65° C. to 190° C., and further such as from 70° C. to 130° C., and even further such as from 80° C. to 105° C.

The film-forming polymer of the compositions may be chosen from any silicone resin that has film forming properties and any other organic material with film forming properties.

In one embodiment, the film-forming silicone resin is chosen from silsesquioxanes and siloxysilicates.

The use of silicone polymers or derivatives as film-forming agents in cosmetic compositions is known in the art. See, e.g., U.S. Pat. Nos. 5,965,112; 5,800,816; 5,911,974; and 5,959,009, the disclosures of which are incorporated by reference herein. Silicone resin nomenclature is known in the art as “MDTQ” nomenclature, whereby a silicone resin is described according to the various monomeric siloxane repeating units which make up the polymer. Each letter of “MDTQ” denotes a different type of unit. The symbol M denotes the monofunctional unit (CH₃)₃SiO_{1/2}. The unit is considered monofunctional because the silicone atom only shares one oxygen for the formation of the chain.

The “M” unit can be represented as:

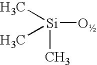

At least one of the methyl groups can be replaced, e.g., to give a unit with formula [R(CH₃)₂]SiO_{/2}, as represented in the following structure:

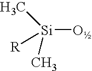

wherein R is other than a methyl group.

The symbol D denotes the difunctional unit (CH₃)₂SiO_{2/2} where two of the available bonds from the silicone atom are used for binding to oxygen for the formation of the polymeric chain. The “D” unit, which is the major building block of dimethicone oils, can be represented as:

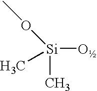

The symbol T denotes the trifunctional unit, (CH₃)SiO_{3/2} and can be represented as

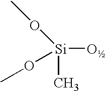

As in the M unit, any methyl group could be replaced in D or T with a group R that is other than methyl. Finally, the symbol Q denotes a quadrifunctional unit SiO_{4/2} and can be represented as:

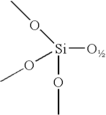

The number of different silicones that can be manufactured is staggering. It would be clear to one skilled in the art that the properties of each of the silicones will vary depending on the type of monomer, the type of substitution, the size of the polymeric chain, and the degree of cross linking or size of the side chain. Different properties are attained depending on whether the backbone is a silicone chain with carbon-based side chains or whether the backbone is carbon-based with silicone side chains.

As described above, the film-forming silicone resin may be chosen from siloxysilicates and silsesquioxanes. Any siloxysilicates or silsesquioxanes that function as a film-former are within the practice of the invention. In one embodiment, the film-forming silicone resin is chosen from substituted siloxysilicates and silsesquioxanes. A substituted siloxysilicate or a substituted silsesquioxane may be, for example, a siloxysilicate or a silsesquioxane where a methyl group has been substituted with a longer carbon chain such as an ethane, propane, or butane. The carbon chain may be saturated or unsaturated.

In one embodiment, the film-forming silicone resin is chosen from siloxysilicates such as trimethylsiloxysilicates, which are represented by the following formula: [(CH₃)₃—Si—O]ₓ—(SiO_{4/2})_{y} (MQ Units) where x and y can have values ranging from 50 to 80. In a further embodiment, a siloxysilicate may be chosen from any combination of M and Q units, such as, for example, [(R)₃—Si—O]ₓ—(SiO_{4/2})_{y}, where R is chosen from a methyl group and longer carbon chains.

In a further embodiment, the film-forming silicone resin is chosen from silsesquioxanes that are represented by the following formula: (CH₃SiO_{3/2})ₓ (T Units) where x has a value of up to several thousand and the CH₃ may be replaced by an R, as described above for T units. In one embodiment, the silsesquioxane is chosen from polymethylsilsesquioxanes, which are silsesquioxanes that do not have a substituent replacing the methyl group. The polymethylsilsesquioxanes useful in the present invention are film-formers and can, for example, have about 500 or less T units, such as from about 50 to about 500 T units. In another embodiment, they have a melting point from about 40° C. to about 80° C. These silicone resins are soluble or dispersible in volatile silicones or other organic liquids. Not all polymethylsilsesquioxanes are film-formers. For example, the highly polymerized polymethylsilsesquioxanes (T Resins), such as Tospearl™ from Toshiba or KMP590 from Shin-Etsu are highly insoluble and therefore are not effective film-formers. The molecular weight of these polymethylsilsesquioxanes is difficult to determine and they generally contain a thousand or more T units.

Other suitable polymethylsilsesquioxanes useful in accordance with the present invention include Belsil PMS MK, also referred to as Resin MK, available from Wacker Chemie. This polymethylsilsesquioxane is a polymer primarily formed of polymerized repeating units of CH₃SiO_{3/2} (T units) and which can also contain up to about 1% (by weight or by mole) of (CH₃)₂SiO_{2/2} (D units). It is believed that the polymers are in a “cage” and “ladder” configuration as exemplified in the figure below. The weight-average molecular weight of the “cage” unit has been calculated to be 536. The majority of the polymer is in the “ladder” configuration, where the ends are capped with ethoxy (CH₃CH₂O) groups. The weight percent of ethoxy present is about 4.5% and the mole percent is about 7% (silicone units). Since this functionality can react with water, a small and variable amount of SiOH can also be present. The weight-average molecular weight can be, for example, from about 500 to about 50,000, such as about 10,000.

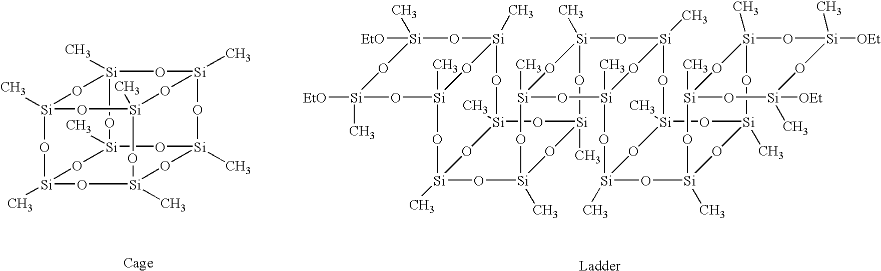

Polymethylsilsesquioxanes suitable for use in the present invention also include KR-220L available from SHIN-ETSU. The structure of KR-220L is made up of mostly silicone T-units (CH₃SiO_{3/2}) with Si—OH or silanol end units. There are no D units. Other polymethylsilsesquioxanes that can be useful in the practice of the invention include KR-242A, which has a structure of about 98% methyl T units and about 2% dimethyl D units with Si—OH or silanol end units, and KR-251, which has a structure of about 88% methyl T units and about 12% dimethyl D units with Si—OH or silanol end units, both of which are available from SHIN-ETSU.

In a further embodiment, the film-forming silicone resin is chosen from combinations of M, D, T, and Q units comprising at least two units chosen from M, D, T, and Q and that satisfy the relationship RₙSiO(_{4-n)/2} wherein n is a value ranging from 1.0 to 1.50. Some resins of this type are disclosed in U.S. Pat. No. 6,074,654, the disclosure of which is incorporated by reference herein. R may be a methyl group or any carbon chain as long as the silicone resin retains its film forming properties. (Up to 5%, of silanol or alkoxy functionality may also be present in the resin structure as a result of processing.) The film-forming silicone resins may be solid at about 25° C. and may have a molecular weight ranging from 1000 to 10000 grams/mole.

In a further embodiment, the film-forming silicone resin comprises repeating M units and Q units. The ratio of M units to Q units may be, for example, 0.7:1. The film-forming silicone resin may be chosen from Wacker 803 and 804 available from Wacker Silicones Corporation and G.E. 1170-002 from General Electric.

In a further embodiment, the film-forming silicone resin is a copolymer wherein at least one unit of the copolymer is chosen from M, D, T, and Q silicone units and at least one additional unit of the copolymer is chosen from an ester. The film-forming silicone resin may be chosen from, for example, diisostearoyl trimethylolpropane siloxysilicates, such as SF 1318, available from GE Silicones.

The film forming polymer may also be a silicone polymer such a high viscosity phenylated silicone such as phenyltrimethicone available as Belsil PDM 1000

The film forming polymer of the compositions according to the present invention may also be a non silicone film former. This non silicone film former may be chosen from, for example, polyethylene; vinylpyrrolidone/vinyl acetate (PVP/VA) copolymers such as the Luviskol® VA grades (all ranges) from BASF® Corporation and the PVP/VA series from ISP; acrylic fluorinated emulsion film formers including Foraperle® film formers such as Foraperle® 303 D from Elf Atochem (although Foraperle® may not be appropriate for some cosmetic formulations); GANEX® copolymers such as butylated PVP, PVP/Hexadecene copolymer, PVP/Eicosene copolymer or tricontanyl; Poly(vinylpyrrolidone/diethylaminoethyl methacrylate) or PVP/Dimethylaminoethylmethacrylate copolymers such as Copolymer 845; Resin ACO-5014 (Imidized lB/MA copolymer); other PVP based polymers and copolymers; alkyl cycloalkylacrylate copolymers (See WO 98/42298, the disclosure of which is hereby incorporated by reference); Mexomere® film formers and other allyl stearate/vinyl acetate copolymers (allyl stearate/VA copolymers); polyolprepolymers such as PPG-12/SMDI copolymer, polyolprepolymers such as PPG-1 2/SM Dl copolymer, Poly(oxy-1,2-ethanediyl), α-hydro-ω-hydroxy-polymer with 1,1′-methylene-bis-(4-isocyanatocyclohexane) available from Barnet; Avalure™ AC Polymers (Acrylates Copolymer) and Avalure™ UR polymers (Polyurethane Dispersions), available from BFGoodrich.

The film former which also may be used within the framework of the invention includes film formers having any film former chemistry known in the art such as: PVP, acrylates, and urethanes; synthetic polymers of the polycondensate type or free-radical type, or ionic type, polymers of natural origin and mixtures thereof or any other film former known within the practice of the cosmetic and pharmaceutical arts which one skilled in the art may determine to be compatible. Film formers that may be used are also disclosed in the International Cosmetic Ingredient Dictionary and Handbook, Vol. 2 (7^{th} ed. 1999), more particularly the emollients disclosed on pages 1636-1638. The disclosure of the International Cosmetic Ingredient Dictionary and Handbook, Vol. 2, pages 1636-1638, is hereby incorporated by reference.

An appropriate concentration of the film former may be determined by one of skill in the art and can vary considerably based on the application. For example, for cosmetic compositions, the film former may be used in an amount from 0.1% to 20% such as, for example, from 1% to 10% by weight, relative to the total weight of the composition.

The concentrations of the film-forming polymer and of the at least one structuring polymer may be chosen according to the desired hardness and desired stability of the compositions and according to the specific application envisaged. The respective concentrations of the at least one structuring polymer and of the at least one solid substance can be such that a disintegrable solid which does not flow under its own weight is obtained.

Depending on the intended application, such as a stick, hardness of the composition may also be considered. The hardness of a composition may, for example, be expressed in grams (g). The composition of the present invention may, for example, have a hardness ranging from 20 g to 2000 g, such as from 20 g to 900 g, and further such as from 20 g to 600 g.

This hardness is measured in one of two ways. A first test for hardness is according to a method of penetrating a probe into said composition and in particular using a texture analyzer (for example TA-XT2 from Rhéo) equipped with an ebonite cylinder of height 25 mm and diameter 8 mm. The hardness measurement is carried out at 200° C. at the center of 5 samples of said composition. The cylinder is introduced into each sample of composition at a pre-speed of 2 mm/s and then at a speed of 0.5 mm/s and finally at a post-speed of 2 mm/s, the total displacement being 1 mm. The recorded hardness value is that of the maximum peak observed. The measurement error is ±50 g.

The second test for hardness is the “cheese wire” method, which involves cutting an 8.1 mm tube of composition and measuring its hardness at 20° C. using a DFGHS 2 tensile testing machine from Indelco-Chatillon Co. at a speed of 100 mm/minute. The hardness value from this method is expressed in grams as the shear force required to cut a stick under the above conditions. According to this method, the hardness of compositions according to the present invention which may be in stick form may, for example, range from 30 g to 300 g, such as from 30 g to 250 g, and further such as from 30 g to 200 g.

The hardness of the composition of the present invention may be such that the compositions are self-supporting and can easily disintegrate to form a satisfactory deposit on a keratinous material. In addition, this hardness may impart good impact strength to the inventive compositions that may be molded or cast, for example, in stick or dish form.

The skilled artisan may choose to evaluate a composition using at least one of the tests for hardness outlined above based on the application envisaged and the hardness desired. If one obtains an acceptable hardness value, in view of the intended application, from at least one of these hardness tests, the composition falls within the scope of the invention.

According to the present invention, the compositions in stick form may also possess the properties of deformable, flexible elastic solids and may also have noteworthy elastic softness upon application to a keratinous material. The compositions in stick form of the prior art do not have this elasticity and flexibility.

The at least one structuring polymer has an affinity with the fatty phase and in particular with a chemical portion of one of the oils forming the liquid fatty phase of the composition so that physical links with the oils, such as hydrogen bonds are formed.

The at least one liquid fatty phase, in one embodiment, may comprise at least one oil. The at least one oil, for example, may be chosen from polar oils and apolar oils including hydrocarbon-based liquid oils and oily liquids at room temperature. In one embodiment, the compositions of the invention comprise at least one structuring polymer and at least one polar oil. The polar oils of the invention, for example, may be added to the apolar oils, the apolar oils acting in particular as co-solvent for the polar oils.

According to the invention, the structuring of the at least one liquid fatty phase may, for example, be obtained with the aid of at least one polymer of formula (I). In general, the polymers of formula (I) may be in the form of mixtures of polymers, these mixtures also possibly containing a synthetic product corresponding to a compound of formula (I) in which n is 0, i.e., a diester.

The liquid fatty phase of the composition may contain more than 30%, for example, more than 40%, of liquid oil(s) containing a group similar to that of the units containing a hetero atom of the structuring polymer, and for example from 50% to 100%. In one embodiment, the liquid fatty phase structured with a polyamide-type skeleton contains a high quantity, i.e., greater than 30%, for example greater than 40% relative to the total weight of the liquid fatty phase, or from 50% to 100%, of at least one apolar, such as hydrocarbon-based, oil. For the purposes of the invention, the expression “hydrocarbon-based oil” means an oil essentially comprising carbon and hydrogen atoms, optionally with at least one group chosen from hydroxyl, ester, carboxyl, and ether groups.

For a liquid fatty phase structured with a polymer containing a partially silicone-based skeleton, this fatty phase may contain more than 30%, for example, more than 40%, relative to the total weight of the liquid fatty phase and, for example, from 50% to 100%, of at least one silicone-based liquid oil, relative to the total weight of the liquid fatty phase.

For a liquid fatty phase structured with an apolar polymer of the hydrocarbon-based type, this fatty phase may contain more than 30%, for example more than 40% by weight, or from 50% to 100% by weight, of at least one liquid apolar, such as hydrocarbon-based, oil, relative to the total weight of the liquid fatty phase.

For example, the at least one polar oil useful in the invention may be chosen from:

hydrocarbon-based plant oils with a high content of triglycerides comprising fatty acid esters of glycerol in which the fatty acids may have varied chain lengths from C₄ to C₂₄, these chains possibly being chosen from linear and branched, and saturated and unsaturated chains; these oils are chosen from, for example, wheat germ oil, corn oil, sunflower oil, karite butter, castor oil, sweet almond oil, macadamia oil, apricot oil, soybean oil, cotton oil, alfalfa oil, poppy oil, pumpkin oil, sesame oil, marrow oil, rapeseed oil, avocado oil, hazelnut oil, grape seed oil, blackcurrant seed oil, evening primrose oil, millet oil, barley oil, quinoa oil, olive oil, rye oil, safflower oil, candlenut oil, passion flower oil and musk rose oil; or alternatively caprylic/capric acid triglycerides such as those sold by Stearineries Dubois or those sold under the names Miglyol 810, 812 and 818 by Dynamit Nobel;

synthetic oils or esters of formula R₅COOR₆ in which R₅ is chosen from linear and branched fatty acid residues containing from 1 to 40 carbon atoms and R₆ is chosen from, for example, a hydrocarbon-based chain containing from 1 to 40 carbon atoms, on condition that R₅+R₆>10, such as, for example, purcellin oil (cetostearyl octanoate), isononyl isononanoate, C₁₂-C₁₅ alkyl benzoates, isopropyl myristate, 2-ethylhexyl palmitate, isostearyl isostearate and alkyl or polyalkyl octanoates, decanoates or ricinoleates; hydroxylated esters such as isostearyl lactate and diisostearyl malate; and pentaerythritol esters;

synthetic ethers containing from 10 to 40 carbon atoms;

C₈ to C₂₆ fatty alcohols such as oleyl alcohol; and

C₈ to C₂₆ fatty acids such as oleic acid, linolenic acid or linoleic acid.

The at least one apolar oil according to the invention is chosen from, for example, silicone oils chosen from volatile and non-volatile, linear and cyclic polydimethylsiloxanes (PDMSs) that are liquid at room temperature; polydimethylsiloxanes comprising alkyl or alkoxy groups which are pendant and/or at the end of the silicone chain, the groups each containing from 2 to 24 carbon atoms; phenylsilicones such as phenyl trimethicones, phenyl dimethicones, phenyl trimethylsiloxy diphenylsiloxanes, diphenyl dimethicones, diphenyl methyldiphenyl trisiloxanes and 2-phenylethyl trimethylsiloxysilicates; hydrocarbons chosen from linear and branched, volatile and non-volatile hydrocarbons of synthetic and mineral origin, such as volatile liquid paraffins (such as isoparaffins and isododecane) or non-volatile liquid paraffins and derivatives thereof, liquid petrolatum, liquid lanolin, polydecenes, hydrogenated polyisobutene such as Parleam®, and squalane; and mixtures thereof. The structured oils, for example those structured with polyamides such as those of formula (I) or the polyurethanes or polyureas or polyurea-urethanes, may be, in one embodiment, apolar oils, such as an oil or a mixture of hydrocarbon oils chosen from those of mineral and synthetic origin, chosen from hydrocarbons such as alkanes such as Parleam® oil, isoparaffins including isododecane, and squalane, and mixtures thereof. These oils may, in one embodiment, be combined with at least one phenylsilicone oil.

The liquid fatty phase, in one embodiment, contains at least one non-volatile oil chosen from, for example, hydrocarbon-based oils of mineral, plant and synthetic origin, synthetic esters or ethers, silicone oils and mixtures thereof.

In practice, the total liquid fatty phase may be present, for example, in an amount ranging from 1% to 99% by weight relative to the total weight of the composition; further examples include ranges of 5 to 95.5%, 10% to 80% and 20% to 75%.

For the purposes of the invention, the expression “volatile solvent or oil” means any non-aqueous medium capable of evaporating on contact with the skin or the lips in less than one hour at room temperature and atmospheric pressure. The volatile solvent(s) of the invention is (are) organic solvents, such as volatile cosmetic oils that are liquid at room temperature, having a non-zero vapor pressure, at room temperature and atmospheric pressure, ranging in particular from 10⁻² to 300 mmHg and, for example, greater than 0.3 mmHg. The expression “non-volatile oil” means an oil which remains on the skin or the lips at room temperature and atmospheric pressure for at least several hours, such as those having a vapor pressure of less than 10⁻² mmHg.

According to the invention, these volatile solvents may facilitate the staying power or long wearing properties of the composition on the skin, the lips or superficial body growths. The solvents can be chosen from hydrocarbon-based solvents, silicone solvents optionally comprising alkyl or alkoxy groups that are pendant or at the end of a silicone chain, and a mixture of these solvents. The volatile oil(s), in one embodiment, is present in an amount ranging from 0% to 95.5% relative to the total weight of the composition, such as from 2% to 75% or, for example, from 10% to 45%. This amount may be determined by a person skilled in the art according to the desired staying power or long wearing properties.

The silicone elastomer powders useful in this invention comprise particles of a globular or spherical core of cured silicone elastomer particle that, in general, have an average particle diameter from 0.1 μm to 100 μm, wherein the core is coated with a silicone resin e.g., a coating layer formed of a polyorganosilsesquioxane resin, which in general, is present in an amount of from 1 to 500 parts by weight per 100 parts by weight of the core silicone elastomer.

In certain embodiments, the silicone elastomer forming the core particles is a cured diorganopolysiloxane having linear diorganopolysiloxane segments represented by the general formula (II):

(R—Si—O)ₐ (II);

wherein each R is, independently from the others, an unsubstituted or substituted monovalent hydrocarbon group having 1 to 20 carbon atoms exemplified by alkyl groups such as methyl, ethyl, propyl and butyl groups, aryl groups such as phenyl and tolyl groups, alkenyl groups such as vinyl and allyl groups and aralkyl groups such as 2-phenylethyl and 2-phenylpropyl groups as well as those substituted hydrocarbon groups obtained by replacing a part or all of the hydrogen atoms in the above named hydrocarbon groups with substituents including halogen atoms, epoxy group, amino group, mercapto group, (meth)acryloxy group and the like such as chloromethyl and 3,3,3-trifluoropropyl groups, at least 90% by moles of the groups R being preferably methyl groups, and the subscript a is a positive integer in the range of from 5 to 5000 such as from 10 to 1000.

The coated silicone elastomer particles can be prepared by in situ hydrolysis and condensation reaction of a trialkoxy silane compound in the presence of the cured silicone elastomer particles in an aqueous dispersion so as to form the coating layer of a silicone e.g., polyorganosilsesquioxane, resin on the surface of the silicone elastomer particles. The method of preparation includes admixing an aqueous dispersion of particles of a cured silicone elastomer having an average particle diameter in the range from 0.1 μm to 100 μm with an alkaline compound and a trialkoxy silane compound represented by the general formula (III):

R′—Si(OR″)₃ (III);

wherein R′ is an unsubstituted or substituted monovalent hydrocarbon group, and R″ is an alkyl group having 1 to 6 carbon atoms, at a temperature not exceeding 60° C., and under agitation. Specific examples of the preparation of such silicone elastomer coated particles are described in U.S. Pat. No. 5,538,793. Examples of commercially available silicone elastomer particles coated with polyorganosilsesquioxane are available from Shin-Etsu and include the KSP-100 series, KSP-200 series and KSP-300 series. These are spherical particles of silicone elastomer coated with silicone resin, wherein the silicone elastomer core can be unfunctionalized for the KSP-100 series, functionalized with fluoroalkyl groups for the KSP-200 and functionalized with phenyl groups in the case of the KSP-300. The ratio of the amount of the silicone elastomer powder to the structuring agent generally ranges from about 0.1 to about 9.0. In some embodiments the ratio is from about 0.5 to about 5.0. In other embodiments, the ratio is from about 1.0 to about 4.0, and in yet other embodiments, the ratio is from about 1.0 to about 3.0.

Swelling agents useful in the present invention include silicone oils chosen from volatile and non-volatile, linear and cyclic polydimethylsiloxanes (PDMSs) that are liquid at room temperature (e.g., cyclomethicones and dimethicones); polydimethylsiloxanes comprising alkyl or alkoxy groups which are pendant and/or at the end of the silicone chain, the groups each containing from 2 to 24 carbon atoms; phenylsilicones such as phenyl trimethicones, phenyl dimethicones, phenyl trimethylsiloxy diphenylsiloxanes, diphenyl dimethicones, diphenyl methyldiphenyl trisiloxanes and 2-phenylethyl trimethylsiloxysilicates, and fluorinated silicones. Some low-viscosity silicone oils useful in the present invention are linear polysiloxanes consisting (except for the terminal groups) of units of formula (IV): [(R)₂—Si—O] in which each of the two substituents denoted “R” independently represents a lower alkyl group (having 1 to 6 Carbons). The degree of polymerization (number of repeating units) of these low-viscosity polysiloxanes may range for example from about 3 to 2000. These low-viscosity silicone oils can be prepared according to known methods, or bought commercially: for example series 47 Silbione oil (RHONE POULENC), series 200 oil (DOW CORNING), SF 96 oil (GENERAL ELECTRIC). The terminal groups are, for example, trimethylsilyl, dimethyl hydroxymethylsilyl or vinyl dimethylsilyl groups.

The swelling agent must be cosmetically acceptable. Aside from that criterion, the choice of swelling agent depends on the chemical nature of the silicone elastomer core. For example, silicone elastomer cores having phenyl substituents (e.g., KSP-300) may be used with swelling agents such as a phenyltrimethicone, and cores having fluoro groups (e.g., KSP-200) may be used with agents such as fluorinated silicones. On the other hand, non-functionalized silicone elastomer cores (e.g., KSP-100) may be used with non-functionalized or functionalized swelling agents. Viscosity of the swelling agent generally varies from about 5 to 100,000 cst (centistokes). Agents having a relatively high viscosity will cause relatively slow swelling of the silicone elastomer powder, and agents having low viscosity will generally cause relatively fast swelling of the elastomer powder. More than one swelling agent may be used. In general, the swelling agent is present in the cosmetic composition in an amount of from about 0.1 to about 90%, and preferably from about 0.1 to about 40% by total weight of the composition. Relative amounts of silicone elastomer powder and swelling agent are determined based on the nature of the cosmetic composition. In general, the swelling agent will cause swelling of the elastomer powder in a range from about 10% of the original volume of the powder, to about 2.5 times or more the original volume (as measured, for example, by visual observation of a phase separation of unabsorbed swelling agent and the silicone elastomer core coated with the resin, in its swollen state). Stated differently, the silicone powder will typically absorb up to about 2.5 times its own weight of the swelling agent. In general, the combined weight of silicone elastomer powder and the swelling agent ranges from about 0.1% to about 99% based on the total weight of the composition.

Sunscreens according to this invention that are chemical absorbers actually absorb harmful ultraviolet radiation. It is well known that chemical absorbers are classified, depending on the type of radiation they protect against, as either UV-A or UV-B absorbers. UV-A absorbers generally absorb radiation in the 320 to 400 nm region of the ultraviolet spectrum. UV-A absorbers include anthranilates, benzophenones, and dibenzoyl methanes. UV-B absorbers generally absorb radiation in the 280 to 320 nm region of the ultraviolet spectrum. UV-B absorbers include p-aminobenzoic acid derivatives, camphor derivatives, cinnamates, and salicylates.

Classifying the chemical absorbers generally as UV-A or UV-B absorbers is accepted within the industry. However, a more precise classification is one based upon the chemical properties of the sunscreens. There are eight major classifications of sunscreen chemical properties that are discussed at length in “Sunscreens—Development, Evaluation and Regulatory Aspects,” by N. Shaath et al., 2nd. Edition, pages 269-273, Marcel Dekker, Inc. (1997). This discussion, in its entirety, is incorporated by reference herein.

The sunscreens that may be formulated according to the present invention typically comprise chemical absorbers, but may also comprise physical blockers. Exemplary sunscreens which may be formulated into the compositions of the present invention are chemical absorbers such as p-aminobenzoic acid derivatives, anthranilates, benzophenones, camphor derivatives, cinnamic derivatives, dibenzoyl methanes (such as avobenzone also known as Parsol®1789), diphenylacrylate derivatives, salicylic derivatives, triazine derivatives, benzimidazole compounds, bis-benzoazolyl derivatives, methylene bis-(hydroxyphenylbenzotriazole) compounds, the sunscreen polymers and silicones, or mixtures thereof. These are variously described in U.S. Pat. Nos. 2,463,264, 4,367,390, 5,166,355 and 5,237,071 and in EP-0,863,145, EP-0,517,104, EP-0,570,838, EP-0,796,851, EP-0,775,698, EP-0,878,469, EP-0,933,376, EP-0,893,119, EP-0,669,323, GB-2,303,549, DE-1,972,184 and WO-93/04665, also expressly incorporated by reference. Also exemplary of the sunscreens which may be formulated into the compositions of this invention are physical blockers such as cerium oxides, chromium oxides, cobalt oxides, iron oxides, red petrolatum, silicone-treated titanium dioxide, titanium dioxide, zinc oxide, and/or zirconium oxide, or mixtures thereof.

A wide variety of sunscreens is described in U.S. Pat. No. 5,087,445, issued to Haffey, et al., on Feb. 11, 1992; U.S. Pat. No. 5,073,372, issued to Turner, et al., on Dec. 17, 1991; and Chapter VIII of Cosmetics and Science and Technology, by Segarin, et al., pages 189 et seq. (1957), all of which are incorporated herein by reference in their entirety.

Sunscreens which may be formulated into the compositions of the instant invention are those selected from among: aminobenzoic acid, amyldimethyl PABA, cinoxate, diethanolamine p-methoxycinnamate, digalloyl trioleate, dioxybenzone, 2-ethoxyethyl p-methoxycinnamate, ethyl 4-bis(hydroxypropyl)aminobenzoate, 2-ethylhexyl-2-cyano-3,3-diphenylacrylate, ethylhexyl p-methoxycinnamate, 2-ethylhexyl salicylate, glyceryl aminobenzoate, homomenthyl salicylate, homosalate, 3-imidazol-4-ylacrylic acid and ethyl ester, methyl anthranilate, octyldimethyl PABA, 2-phenylbenzimidazole-5-sulfonic acid and salts, red petrolatum, sulisobenzone, titanium dioxide, triethanolamine salicylate, N,N,N-trimethyl-4-(2-oxoborn-3-ylidene methyl)anillinium methyl sulfate, and mixtures thereof.

Sunscreens active in the UV-A and/or UV-B range can also include:

p-aminobenzoic acid,

oxyethylene (25 mol) p-aminobenzoate,

2-ethylhexyl p-dimethylaminobenzoate,

ethyl N-oxypropylene p-aminobenzoate,

glycerol p-aminobenzoate,

4-isopropylbenzyl salicylate,

2-ethylhexyl 4-methoxycinnamate,

methyl diisopropylcinnamate,

isoamyl 4-methoxycinnamate,

diethanolamine 4-methoxycinnamate,

3-(4′-trimethylammunium)-benzyliden-bornan-2-one methylsulfate,

2-hydroxy-4-methoxybenzophenone,

2-hydroxy-4-methoxybenzophenone-5-sulfonate,

2,4-dihydroxybenzophenone,

2,2′,4,4′-tetrahydroxybenzophenone,

2,2′-dihydroxy-4,4′dimethoxybenzophenone,

2-hydroxy-4-n-octoxybenzophenone,

2-hydroxy-4-methoxy-4′-methoxybenzophenone,

-(2-oxoborn-3-ylidene)-tolyl-4-sulfonic acid and soluble salts thereof,

3-(4′-sulfo)benzyliden-bornan-2-one and soluble salts thereof,

3-(4′methylbenzylidene)-d,1-camphor,

3-benzylidene-d,1-camphor,

benzene 1,4-di(3-methylidene-10-camphosulfonic) acid and salts thereof (the product Mexoryl SX described in U.S. Pat. No. 4,585,597 issued to Lange, et al., on Apr. 29, 1986),

urocanic acid,

2,4,6-tris[p-(2′-ethylhexyl-1′-oxycarbonyl)-anilino]-1,3,5-triazine,

2-[(p-(tertiobutylamido)anilino]-4,6-bis-[(p-(2′-ethylhexyl-1′-oxycarbonyl)anilino]-1,3,5-triazine,

2,4-bis{[4-(2-ethyl-hexyloxy)]-2-hydroxy]-phenyl}-6-(4-methoxy-phenyl)-1,3,5-triazine (“TINOSORB S” marketed by Ciba),

the polymer of N-(2 et 4)-[(2-oxoborn-3-yliden)methyl]benzyl]-acrylamide,

1,4-bisbenzimidazolyl-phenylen-3,31,5,51-tetrasulfonic acid and salts thereof,

the benzalmalonate-substituted polyorganosiloxanes,

the benzotriazole-substituted polyorganosiloxanes (Drometrizole Trisiloxane),

dispersed 2,21-methylene-bis-[6-(2H-benzotriazol-2-yl)-4-(1,1,3,3-tetramethylbutyl)phenol] such as that marketed under the trademark MIXXIM BB/100 by Fairmount Chemical, or micronized in dispersed form thereof such as that marketed under the trademark TINOSORB M by Ciba-Geigy, and

solubilized 2,2′-methylene-bis-[6-(2H-benzotriazol-2-yl)-4-(methyl)phenol] such as that marketed under the trademark MIXXIM BB/200 by Fairmount Chemical.

Typically combinations of one of more of these sunscreens are used.

The dibenzoyl methane derivatives other than avobenzone are described, for example, in FR-2,326,405, FR-2,440,933 and EP-0,114,607, hereby expressly incorporated by reference.

Other dibenzoyl methane sunscreens other than avobenzone include (whether singly or in any combination):

2-methyldibenzoylmethane

4-methyldibenzoylmethane

4-isopropyldibenzoylmethane

4-tert.-butyldibenzoylmethane

2,4-dimethyldibenzoylmethane

2,5-dimethyldibenzoylmethane

4,4′-diisopropyldibenzoylmethane

4,4′-dimethoxydibenzoylmethane

2-methyl-5-isopropyl-4′-methoxydibenzoylmethane

2-methyl-5-tert.-butyl-4′-methoxydibenzoylmethane

2,4-dimethyl-4′-methoxydibenzoylmethane

2,6-dimethyl-4-tert.-butyl-4′-methoxydibenzoylmethane

Additional sunscreens that can be used are described in pages 2954-2955 of the International Cosmetic Ingredient Dictionary and Handbook (9^{th} ed. 2002).

The at least one liquid fatty phase of the compositions of the invention may further comprise a dispersion of lipid vesicles. The compositions of the invention may, for example, be in the form of a fluid simple emulsion, a fluid multiple emulsion, a rigid simple emulsion or a rigid multiple emulsion. The simple emulsion or multiple emulsion may comprise a continuous phase chosen from an aqueous phase optionally containing dispersed lipid vesicles or oil droplets, or a fatty phase optionally containing dispersed lipid vesicles or water droplets. In one embodiment, the composition has a continuous oily phase or fatty phase, may contain less than 10% by weight of water and be in the form of a stick or a dish.

The compositions of the invention may further comprise at least one additional fatty material. The at least one additional fatty material may, for example, be chosen from gums, fatty materials pasty at ambient temperature, and resins.

The compositions of the invention may further include formulation aids that are usually employed in the field of application envisaged. The formulation aids used in the present invention can be, but are not limited to, fatty substances. Useful fatty substances include, but are not limited to, organic and organosilicone emulsifiers for water-in-oil systems. Examples of organic emulsifiers include any ethoxylated surfactants known in the art such as Polysorbate-20, Laureth-7, Laureth-4, Sepigel® 305 available from SEPPIC and other similar ingredients disclosed in the International Cosmetic Ingredient Dictionary and Handbook, Vol. 4 (9^{th} ed. 2002), more particularly the emulsifiers disclosed on pages 2962-2971. The disclosure of the International Cosmetic Ingredient Dictionary and Handbook, Vol. 4, pages 2962-2971, is hereby incorporated by reference. Examples of organosilicone emulsifiers include cetyl dimethicone copolyol-polyglyceryl-4-isostearate-hexylaurate (ABIL® WE 09) available from Goldschmidt Chemical Corporation, Cetyl Dimethicone Copolyol (ABIL® EM 90), (ABIL® EM 97), Laurylmethicone Copolyol (5200), Cyclomethicone (and) Dimethicone Copolyol (DC 5225 C and DC 3225 C) available from GE Silicones, Cyclopentasiloxane & Dimethicone Copolyol (GE SF 1528) or any other formulation aids known by one of skill in the art. Other fatty substances useful as formulation aids include but are not limited to, silicones in esterified or unesterified liquid form or in esterified solid form, such as behenate dimethicone; and non-silicone fatty substances including oils such as vegetable and mineral oil; animal and/or synthetic waxes such as beeswax, parafin, rice bran wax, candelilla wax, carnauba wax and derivatives thereof; and hydrocarbon gels or bentone type gels, such as Gel SS71, Gel EA2786, Quaternium-18 Bentonite, 38 CE, Gel ISD V or Gel ISD.

These substances may be selected variously by the person skilled in the art in order to prepare a composition that has the desired properties, for example, consistency or texture.

Plasticizers may also be added to the compositions to improve the flexibility and cosmetic properties of the resulting formulation. Plasticizers are materials that soften synthetic polymers. They are frequently required to avoid brittleness and cracking of film formers. One skilled in the art may routinely vary the amount of plasticizer desired based on the properties desired and the application envisaged. Plasticizers useful in the practice of the invention include lecithin, polysorbates, dimethicone copolyol, glycols, citrate esters, glycerin, dimethicone, and other similar ingredients disclosed in the International Cosmetic Ingredient Dictionary and Handbook, Vol. 4 (9^{th} ed. 2002), more particularly the plasticizers disclosed on page 2927. The disclosure of the International Cosmetic Ingredient Dictionary and Handbook, Vol. 4, page 2927, is hereby incorporated by reference.

The composition of the present invention may also further comprise at least one suitable additive commonly used in the field concerned chosen from coloring agents, antioxidants, essential oils, preserving agents, fragrances, fillers, pasty fatty substances, waxy fatty substances, neutralizing agents, liposoluble polymers, and cosmetically active agents and dermatological active agents such as, for example, emollients, moisturizers, vitamins and essential fatty acids. The compositions of the invention may also be optionally thickened with an aqueous-phase thickener or gelled with a gelling agent and/or containing ingredients soluble in water.

In one embodiment, a suitable additive is chosen from a wax. As used herein, a “wax” may be any lipophilic fatty compound. Non-limiting examples of such waxes include waxes of natural origin, such as beeswax, carnauba wax, candelilla wax, ouricury wax, Japan wax, cork fiber wax, sugar cane wax, paraffin waxes, lignite wax, microcrystalline waxes, lanolin wax, montan wax and ozokerites, hydrogenated oils such as hydrogenated jojoba oil, jojoba esters, waxes of synthetic origin, such as polyethylene waxes derived from polymerization of ethylene, waxes obtained by Fischer-Tropsch synthesis, fatty acid esters and glycerides, and silicone waxes such as derivatives of poly(di)methylsiloxane. The wax, in one embodiment, is present in an amount ranging from 0.5% to 15% relative to the total weight of the composition, such as from 0.1% to 10%.

Another embodiment of the invention relates to a skin lip, or keratinous fiber care or make-up composition comprising a structured composition containing at least one liquid fatty phase structured with at least one structuring polymer comprising a polymer skeleton comprising at least one hydrocarbon-based repeating unit comprising at least one hetero atom, at least one sunscreen agent, a silicone elastomer powder comprising a silicone elastomer core coated with a silicone resin, and a swelling agent for the powder.

Additionally, an embodiment of the invention relates to a skin, lip, or keratinous fiber care or make-up composition comprising a structured composition containing at least one liquid fatty phase structured with at least one structuring polymer comprising a polymer skeleton comprising at least one hydrocarbon-based repeating unit comprising at least one hetero atom, at least one sunscreen agent, at least one coloring agent, a silicone elastomer powder comprising a silicone elastomer core coated with a silicone resin, and a swelling agent for the powder.

Additionally, an embodiment of the invention relates to a method of making up skin, lips, or keratinous fibers or caring for skin, lips, or keratinous fibers comprising applying to said skin, lips, or keratinous fibers a structured composition containing at least one liquid fatty phase structured with at least one structuring polymer comprising a polymer skeleton comprising at least one hydrocarbon-based repeating unit comprising at least one hetero atom, at least one sunscreen agent, a silicone elastomer powder comprising a silicone elastomer core coated with a silicone resin, and a swelling agent for the powder.

Needless to say, the person skilled in the art will take care to select the optional additional additives and the amount thereof such that not all of the possible advantageous properties of the composition according to the invention are, or are substantially, adversely affected by the addition(s) envisaged.

The transfer resistance of a composition may be evaluated by a transfer resistance test. A composition is applied to the clean, bare skin of a group of subjects as a smooth, thin layer, such as a 1×1″ square in size. The composition is allowed to air dry for 5 minutes and using a facial tissue or other cloth or sponge and medium pressure the area is wiped as if attempting to wipe the product off of the skin. The subjects are then asked to evaluate the results. Positive results are described as the presence of a makeup film that could not be easily removed without soap and water. For example, the results may be rated on a scale of 1 to 5 with 5 being the best and 1 being the worst.

The packaging and application device for any subject of the invention may be chosen and manufactured by persons skilled in the art on the basis of their general knowledge, and adapted according to the nature of the composition to be packaged. Indeed, the type of device to be used can be in particular linked to the consistency of the composition, in particular to its viscosity; it can also depend on the nature of the constituents present in the composition, such as the presence of volatile compounds.

The present invention is also directed to a cosmetic process for caring for, making up or treating a keratinous material, such as that of a human being, and further such as human skin, lips, or keratinous fibers, comprising the application to a keratinous material of a cosmetic composition comprising at least one polyamide polymer comprising a polyamide skeleton. The polyamide skeleton comprises at least one end group with at least one chain chosen from alkyl chains comprising at least four carbon atoms and alkenyl chains comprising at least four carbon atoms, bonded to the at least one polyamide skeleton via at least one linking group. The polyamide skeleton may further comprise at least one pendant group with at least one chain chosen from alkyl chains comprising at least four carbon atoms and alkenyl chains comprising at least four carbon atoms, bonded to any carbon or nitrogen of the polyamide skeleton via at least one linking group. The composition also contains at least one sunscreen agent, a silicone elastomer powder comprising a silicone elastomer core coated with a silicone resin, and a swelling agent for the powder.

In another embodiment, the present invention is directed to a process of making a cosmetic composition in the form of a physiologically acceptable composition comprising including in said composition at least one polyamide polymer comprising a polyamide skeleton. The polyamide skeleton comprises at least one end group with at least one chain chosen from alkyl chains comprising at least four carbon atoms and alkenyl chains comprising at least four carbon atoms, bonded to the at least one polyamide skeleton via at least one linking group. The polyamide skeleton may further comprise at least one pendant group with at least one chain chosen from alkyl chains comprising at least four carbon atoms and alkenyl chains comprising at least four carbon atoms, bonded to any carbon or nitrogen of the polyamide skeleton via at least one linking group. The composition also comprises at least one sunscreen agent, a silicone elastomer powder comprising a silicone elastomer core coated with a silicone resin, and a swelling agent for the powder.

The invention will be illustrated by, but is not intended to be limited to, the following examples.

### EXAMPLES

### Examples 1 and 2: SPF 15 Foundation

Example 1 is the comparative example not containing the hetero polymer and example 2 contains the hetero polymer.

| | | | 1% | 2% |
|---|---|---|---|---|
| Phase | Trade Name | INCI Name | w/w | w/w |
| A1 | Uniclear | Polyamide | 0 | 4.10 |
| | 100VG | | | |
| | Octyl | Octyl dodecanol | 3.96 | 3.80 |
| | dodecanol | | | |
| | Finsolv TN | C12-C15 Benzoate | 3.13 | 3.00 |
| | Parsol MCX | Ethylhexyl | 4.17 | 4.00 |
| | | Methoxycinnamate | | |
| | Wickenol | Isononyl Isononanoate | 7.30 | 7.00 |
| | 151 | | | |
| A2 | | Coated pigments | 12.51 | 12 |
| A3 | Arlacel | PEG-30 | 2.09 | 2.00 |
| 3 | P135 | Dihydroxylstearate | | |
| | | Retinyl Palmitate | 0.31 | 0.30 |
| A4 | DC 200 | Dimethicone | 7.30 | 7.00 |
| | Fluid 10 | | | |
| | cst | | | |
| | KSP-100 | Dimethicone/Vinyl | 3.13 | 3.00 |
| | | dimethicone | | |
| | | crosspolymer | | |
| A5 | Butylparaben | Butylparaben | 0.31 | 0.30 |
| | Bentone 38V | Disteardimonium | 1.25 | 1.20 |
| | | Hectorite | | |
| A6 | DC 245 | Cyclopentasiloxane | 5.21 | 5.00 |
| | Fluid | | | |
| 7A | GANZPEARL | Fillers | 5.21 | 5.00 |
| | GMX-0610 | | | |
| A8 | PERMETHYL | Isododecane | 10.43 | 10.00 |
| | 99A | | | |
| B | Water | Water | 30.24 | 29.00 |
| | Glycerine | Glycerine | 2.09 | 2.00 |
| | Magnesium | Magnesium Sulfate | 0.52 | 0.50 |
| | Sulfate | | | |
| | Methylparaben | Methylparaben | 0.21 | 0.20 |
| | Phenoxyethanol | Phenoxyethanol | 0.63 | 0.60 |
| | | Total: | 100.00 | 100.00 |

SPF of comparative example 1: 15.89

SPF of inventive example 2: 19.34

SPF values are an average of SPF values as measured on 5 subjects.

All publications cited in the specification, both patent publications and non-patent publications, are indicative of the level of skill of those skilled in the art to which this invention pertains. All these publications are herein incorporated by reference to the same extent as if each individual publication were specifically and individually indicated as being incorporated by reference.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

What is claimed is:

1. A composition in the form of an emulsion comprising at least one liquid fatty phase which comprises:
(i) at least one structuring polymer comprising:
a polymer skeleton which comprises at least one hydrocarbon-based repeating unit comprising at least one hetero atom;
(ii) at least one sunscreen agent;
(iii) a silicone elastomer powder comprising a silicone elastomer core coated with a silicone resin; and
(iv) a swelling agent for said powder.

2. The composition according to claim 1, wherein said at least one structuring polymer further comprises at least one of:
at least one terminal fatty chain chosen from alkyl chains and alkenyl chains, wherein said at least one terminal fatty chain is bonded to said polymer skeleton via at least one linking group; and
at least one pendant fatty chain chosen from alkyl chains and alkenyl chains, wherein said at least one pendant fatty chain is bonded to said polymer skeleton via at least one linking group.

3. The composition according to claim 2, wherein said alkyl chains and said alkenyl chains each comprise at least four carbon atoms.

4. The composition according to claim 3, wherein said alkyl chains and said alkenyl chains each comprise from 8 to 120 carbon atoms.

5. The composition according to claim 4, wherein said alkyl chains and said alkenyl chains each comprise from 12 to 68 carbon atoms.

6. The composition according to claim 2, wherein said at least one linking group is chosen from single bonds and urea, urethane, thiourea, thiourethane, thioether, thioester, ester, ether and amine groups.

7. The composition according to claim 6, wherein said at least one linking group is an ester group present in a proportion ranging from 15% to 40% of the total number of all ester and hetero atom groups in the at least one structuring polymer.

8. The composition according to claim 7, wherein said at least one linking group is an ester group present in a proportion ranging from 20% to 35% of the total number of all ester and hetero atom groups in the at least one structuring polymer.

9. The composition according to claim 2, wherein said at least one terminal fatty chain is functionalized.

10. The composition according to claim 2, wherein said at least one pendant fatty chain is functionalized.

11. The composition according to claim 2, wherein in said at least one structuring polymer, the percentage of the total number of fatty chains ranges from 40% to 98% relative to the total number of all repeating units and fatty chains in the at least one structuring polymer.

12. The composition according to claim 11, wherein in said at least one structuring polymer, the percentage of the total number of fatty chains ranges from 50% to 95% relative to the total number of all repeating units and fatty chains in the at least one structuring polymer.

13. The composition according to claim 1, wherein said at least one structuring polymer has a weight-average molecular mass of less than 100,000.

14. The composition according to claim 13, wherein said at least one structuring polymer has a weight-average molecular mass of less than 50,000.

15. The composition according to claim 14, wherein said at least one structuring polymer has a weight-average molecular mass ranging from 1000 to 30,000.

16. The composition according to claim 15, wherein said at least one structuring polymer has a weight-average molecular mass ranging from 2000 to 20,000.

17. The composition according to claim 16, wherein said at least one structuring polymer has a weight-average molecular mass ranging from 2000 to 10,000.

18. The composition according to claim 1, wherein said at least one hydrocarbon based repeating unit comprises from 2 to 80 carbon atoms.

19. The composition according to claim 18, wherein said at least one hydrocarbon based repeating unit comprises from 2 to 60 carbon atoms.

20. The composition according to claim 1, wherein said at least one hydrocarbon based repeating unit is chosen from saturated and unsaturated hydrocarbon-based units which are chosen from linear hydrocarbon-based repeating units, branched hydrocarbon-based repeating units and cyclic hydrocarbon-based repeating units.

21. The composition according to claim 1, wherein said at least one hetero atom of said at least one hydrocarbon-based repeating unit is chosen from nitrogen, sulphur, and phosphorus.

22. The composition according to claim 21, wherein said at least one hetero atom is a nitrogen atom.

23. The composition according to claim 21, wherein said at least one hetero atom is combined with at least one atom chosen from oxygen and carbon to form a hetero atom group.

24. The composition according to claim 23, wherein said at least one hetero atom group further comprises a carbonyl group.

25. The composition according to claim 23, wherein said at least one hetero atom group is chosen from amide groups, carbamate groups, and urea groups.

26. The composition according to claim 25, wherein said at least one hetero atom group is an amide group and said polymer skeleton is a polyamide skeleton.

27. The composition according to claim 25, wherein said at least one hetero atom group is chosen from carbamate groups and urea groups and said polymer skeleton is chosen from a polyurethane skeleton, a polyurea skeleton and a polyurethane-polyurea skeleton.

28. The composition according to claim 1, wherein said at least one structuring polymer is chosen from polyamide polymers of formula (I): 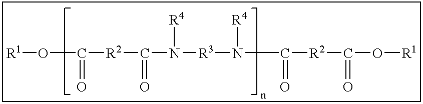
in which:
n is an integer which represents the number of amide units such that the number of ester groups present in said at least one polyamide polymer ranges from 10% to 50% of the total number of all ester groups and all amide groups comprised in said at least one polyamide polymer;
R¹, which are identical or different, are each chosen from alkyl groups comprising at least 4 carbon atoms and alkenyl groups comprising at least 4 carbon atoms;
R², which are identical or different, are each chosen from C₄ to C₄₂ hydrocarbon-based groups with the proviso that at least 50% of all R² are chosen from C₃₀ to C₄₂ hydrocarbon-based groups;
R³, which are identical or different, are each chosen from organic groups comprising atoms chosen from carbon atoms, hydrogen atoms, oxygen atoms and nitrogen atoms, with the proviso that R³ comprises at least 2 carbon atoms; and
R⁴, which are identical or different, are each chosen from hydrogen atoms, C₁ to C₁₀ alkyl groups and a direct bond to at least one group chosen from R³ and another R⁴ such that when said at least one group is chosen from another R⁴, the nitrogen atom to which both R³ and R⁴ are bonded forms part of a heterocyclic structure defined in part by R⁴—N—R³, with the proviso that at least 50% of all R⁴ are chosen from hydrogen atoms.

29. The composition according to claim 28, wherein in said formula (I), n is an integer ranging from 1 to 5.

30. The composition according to claim 29, wherein in said formula (I), n is an integer ranging from 3 to 5.

31. The composition according to claim 28, wherein in said formula (I), said alkyl groups of R¹ and said alkenyl groups of R¹ each independently comprise from 4 to 24 carbon atoms.

32. The composition according to claim 31, wherein in said formula (I), R¹, which are identical or different, are each chosen from C₁₂ to C₂₂ alkyl groups.

33. The composition according to claim 32, wherein in said formula (I), R¹, which are identical or different, are each chosen from C₁₆ to C₂₂ alkyl groups.

34. The composition according to claim 28, wherein in said formula (I), R², which are identical or different, are each chosen from C₁₀ to C₄₂ hydrocarbon based groups with the proviso that at least 50% of all R² are chosen from C₃₀ to C₄₂ hydrocarbon based groups.

35. The composition according to claim 34, wherein at least 75% of all R², which are identical or different, are chosen from C₃₀ to C₄₂ hydrocarbon based groups.

36. The composition according to claim 28, wherein in said formula (I), R³, which can be identical or different, are each chosen from C₂ to C₃₆ hydrocarbon-based groups and polyoxyalkylene groups.

37. The composition according to claim 36, wherein R³, which can be identical or different, are each chosen from C₂ to C₁₂ hydrocarbon-based groups.

38. The composition according to claim 37, wherein in said formula (I), R⁴, which can be identical or different, are each chosen from hydrogen atoms.

39. The composition according to claim 28, wherein said at least one polymer of formula (I) is in the form of a mixture of polymers, wherein said mixture optionally also comprises a compound of formula (I) wherein n is equal to zero.

40. The composition according to claim 1, wherein said at least one structuring polymer has a softening point greater than 50° C.

41. The composition according to claim 40, wherein said at least one structuring polymer has a softening point ranging from 65° C. to 190° C.

42. The composition according to claim 41, wherein said at least one structuring polymer has a softening point ranging from 70° C. to 130° C.

43. The composition according to claim 42, wherein said at least one structuring polymer has a softening point ranging from 80° C. to 105° C.

44. The composition according to claim 1, wherein said at least one structuring polymer is present in the composition in an amount ranging from 0.5% to 80% by weight relative to the total weight of the composition.

45. The composition according to claim 44, wherein said at least one structuring polymer is present in the composition in an amount ranging from 2% to 60% by weight relative to the total weight of the composition.

46. The composition according to claim 45, wherein said at least one structuring polymer is present in the composition in an amount ranging from 5% to 40% by weight relative to the total weight of the composition.

46. The composition according to claim 1, wherein said at least one liquid fatty phase of the composition comprises at least one oil.

47. The composition according to claim 46, wherein said at least one oil is chosen from at least one polar oil and at least one apolar oil.

48. The composition according to claim 47, wherein said at least one polar oil is chosen from:
hydrocarbon-based plant oils with a high content of triglycerides comprising fatty acid esters of glycerol in which the fatty acids comprise chains having from 4 to 24 carbon atoms, said chains optionally being chosen from linear and branched, and saturated and unsaturated chains;
synthetic oils or esters of formula R₅COOR₆ in which R₅ is chosen from linear and branched fatty acid residues comprising from 1 to 40 carbon atoms and R₅+R₆>10;
synthetic ethers containing from 10 to 40 carbon atoms;
C₈ to C₂₆ fatty alcohols; and
C₈ to C₂₆ fatty acids.

49. The composition according to claim 47, wherein said at least one apolar oil is chosen from:
silicone oils chosen from volatile and non-volatile, linear and cyclic polydimethylsiloxanes that are liquid at room temperature;
polydimethylsiloxanes comprising alkyl or alkoxy groups which are pendant and/or at the end of the silicone chain, the groups each containing from 2 to 24 carbon atoms;
phenylsilicones; and
hydrocarbons chosen from linear and branched, volatile and non-volatile hydrocarbons of synthetic and mineral origin.

50. The composition according to claim 1, wherein said at least one liquid fatty phase comprises at least one non-volatile oil.

51. The composition according to claim 50, wherein said at least one non-volatile oil is chosen from hydrocarbon-based oils of mineral, plant and synthetic origin, synthetic esters and ethers, and silicone oils.

52. The composition according to claim 1, wherein said at least one liquid fatty phase is present in an amount ranging from 1% to 99% by weight relative to the total weight of the composition.

53. The composition according to claim 52, wherein said at least one liquid fatty phase is present in an amount ranging from 5% to 95.5% by weight relative to the total weight of the composition.

54. The composition according to claim 52, wherein said at least one liquid fatty phase is present in an amount ranging from 10% to 80% by weight relative to the total weight of the composition.

55. The composition according to claim 52, wherein said at least one liquid fatty phase is present in an amount ranging from 20% to 75% by weight relative to the total weight of the composition.

56. The composition according to claim 1, wherein said at least one liquid fatty phase comprises at least one volatile solvent chosen from hydrocarbon-based solvents and silicone solvents optionally comprising alkyl or alkoxy groups that are pendant or at the end of a silicone chain.

57. The composition according to claim 56, wherein said at least one volatile solvent is present in an amount up to 95.5% relative to the total weight of the composition.

58. The composition according to claim 57, wherein said at least one volatile solvent is present in an amount ranging from 2% to 75% relative to the total weight of the composition.

59. The composition according to claim 58, wherein said at least one volatile solvent is present in an amount ranging from 10% to 45% relative to the total weight of the composition.

60. The composition according to claim 1, wherein said composition further comprises at least one additional fatty material.

61. The composition according to claim 60, wherein said at least one additional fatty material is chosen from gums, fatty materials pasty at ambient temperature, and resins.

62. The composition according to claim 1 further comprising at least one film forming polymer.

63. The composition according to claim 1, wherein said film-forming polymer is present in the composition in an amount ranging from 0.1% to 20% by weight relative to the total weight of the composition.

64. The cosmetic composition of claim 1, wherein said swelling agent comprises linear or cyclic polydimethylsiloxane.

65. The cosmetic composition of claim 64, wherein said polydimethylsiloxane comprises a cyclomethicone.

66. The cosmetic composition of claim 64, wherein said polydimethylsiloxane comprises a dimethicone.

67. The cosmetic composition of claim 1 wherein said swelling agent comprises a phenylmethicone.

68. The cosmetic composition of claim 1 wherein said swelling agent comprises a fluorinated silicone.

69. The cosmetic composition of claim 1, wherein said silicone resin comprises a polyorganosilsesquioxane.

70. The cosmetic composition of claim 1, wherein said silicone elastomer core is unfunctionalized.

71. The cosmetic composition of claim 1, wherein said silicone elastomer core contains pendant functional groups.

72. The cosmetic composition of claim 71, wherein said functional groups comprise fluoroalkyl groups.

73. The cosmetic composition of claim 71, wherein said functional groups comprise phenyl groups.

74. The cosmetic composition of claim 1, wherein said structural agent comprises a polyamide bonded to a fatty chain via an ester group, said swelling agent comprises a dimethicone, and said silicone resin comprises a polyorganosilsesquioxane.

75. The cosmetic composition of claim 1, wherein ratio of amount of said silicone elastomer powder to said structuring agent is from about 0.1 to about 9.0.

76. The cosmetic composition of claim 75, wherein the ratio is from about 0.5 to about 5.0.

77. The cosmetic composition of claim 75, wherein the ratio is from about 1.0 to about 4.0.

78. The cosmetic composition of claim 75, wherein the ratio is from about 1.0 to about 3.0.

79. The composition according to claim 1, wherein the composition is in a form chosen from a fluid simple emulsion, rigid simple emulsion, fluid multiple emulsion, and rigid multiple emulsion.

80. The composition according to claim 1, wherein said composition is a solid.

81. The composition according to claim 80, wherein said composition is a solid chosen from molded and poured sticks.

82. A composition in the form of an emulsion comprising at least one liquid fatty phase which comprises:
(i) at least one structuring polymer, wherein said at least one structuring polymer is at least one polyamide polymer comprising:
a polymer skeleton which comprises at least one amide repeating unit;
(ii) at least one sunscreen agent;
(iii) a silicone elastomer powder comprising a silicone elastomer core coated with a silicone resin; and
(iv) a swelling agent for said powder.

83. The composition according to claim 82, wherein said at least one polyamide polymer further comprises at least one of:
at least one terminal fatty chain chosen from alkyl chains and alkenyl chains, wherein said at least one terminal fatty chain is bonded to said polymer skeleton via at least one linking group; and
at least one pendant fatty chain chosen from alkyl chains and alkenyl chains, wherein said at least one pendant fatty chain is bonded to said polymer skeleton via at least one linking group.

84. The composition according to claim 83, wherein said alkyl chains and said alkenyl chains each comprise at least four carbon atoms.

85. The composition according to claim 84, wherein said alkyl chains and said alkenyl chains each comprise from 8 to 120 carbon atoms.

86. The composition according to claim 85, wherein said alkyl chains and said alkenyl chains each comprise from 12 to 68 carbon atoms.

87. The composition according to claim 83, wherein said at least one linking group is chosen from single bonds and urea, urethane, thiourea, thiourethane, thioether, thioester, ester, ether and amine groups.

88. The composition according to claim 87, wherein said at least one linking group is an ester group present in a proportion ranging from 15% to 40% of the total number of all ester and amide groups in the at least one polyamide polymer.

89. The composition according to claim 88, wherein said at least one linking group is an ester group present in a proportion ranging from 20% to 35% of the total number of all ester and amide groups in the at least one polyamide polymer.

90. The composition according to claim 83, wherein said at least one terminal fatty chain is functionalized.

91. The composition according to claim 83, wherein said at least one pendant fatty chain is functionalized.

92. The composition according to claim 83, wherein in said at least one polyamide polymer, the percentage of the total number of fatty chains ranges from 40% to 98% relative to the total number of all amide units and fatty chains in the at least one polyamide polymer.

93. The composition according to claim 92, wherein in said at least one polyamide polymer, the percentage of the total number of fatty chains ranges from 50% to 95% relative to the total number of all amide units and fatty chains in the at least one polyamide polymer.

94. The composition according to claim 82, wherein said at least one polyamide polymer has a weight-average molecular mass of less than 100,000.

95. The composition according to claim 94, wherein said at least one polyamide polymer has a weight-average molecular mass of less than 50,000.

96. The composition according to claim 95, wherein said at least one polyamide polymer has a weight-average molecular mass ranging from 1000 to 30,000.

97. The composition according to claim 96, wherein said at least one polyamide polymer has a weight-average molecular mass ranging from 2000 to 20,000.

98. The composition according to claim 97, wherein said at least one polyamide polymer has a weight-average molecular mass ranging from 2000 to 10,000.

99. The composition according to claim 82, wherein said at least one polyamide polymer is chosen from polyamide polymers of formula (I): 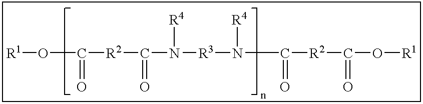
in which:
n is an integer which represents the number of amide units such that the number of ester groups present in said at least one polyamide polymer ranges from 10% to 50% of the total number of all ester groups and all amide groups comprised in said at least one polyamide polymer;
R¹, which are identical or different, are each chosen from alkyl groups comprising at least 4 carbon atoms and alkenyl groups comprising at least 4 carbon atoms;
R², which are identical or different, are each chosen from C₄ to C₄₂ hydrocarbon-based groups with the proviso that at least 50% of all R² are chosen from C₃₀ to C₄₂ hydrocarbon-based groups;
R³, which are identical or different, are each chosen from organic groups comprising atoms chosen from carbon atoms, hydrogen atoms, oxygen atoms and nitrogen atoms with the proviso that R³ comprises at least 2 carbon atoms; and
R⁴, which are identical or different, are each chosen from hydrogen atoms, C₁ to C₁₀ alkyl groups and a direct bond to at least one group chosen from R³ and another R⁴ such that when said at least one group is chosen from another R⁴, the nitrogen atom to which both R³ and R⁴ are bonded forms part of a heterocyclic structure defined in part by R⁴—N—R³, with the proviso that at least 50% of all R⁴ are chosen from hydrogen atoms.

100. The composition according to claim 99, wherein in said formula (I), n is an integer ranging from 1 to 5.

101. The composition according to claim 99, wherein in said formula (I), n is an integer ranging from 3 to 5.

102. The composition according to claim 99, wherein in said formula (I), said alkyl groups of R¹ and said alkenyl groups of R¹ each independently comprise from 4 to 24 carbon atoms.

103. The composition according to claim 102, wherein in said formula (I), R¹, which are identical or different, are each chosen from C₁₂ to C₂₂ alkyl groups.

104. The composition according to claim 103, wherein in said formula (I), R¹, which are identical or different, are each chosen from C₁₆ to C₂₂ alkyl groups.

105. The composition according to claim 99, wherein in said formula (I), R², which are identical or different, are each chosen from C₁₀ to C₄₂ hydrocarbon based groups with the proviso that at least 50% of all R² are chosen from C₃₀ to C₄₂ hydrocarbon based groups.

106. The composition according to claim 105, wherein at least 75% of all R², which are identical or different, are chosen from C₃₀ to C₄₂ hydrocarbon based groups.

107. The composition according to claim 106, wherein in said formula (I), R³, which can be identical or different, are each chosen from C₂ to C₃₆ hydrocarbon-based groups and polyoxyalkylene groups.

108. The composition according to claim 107, wherein R³, which can be identical or different, are each chosen from C₂ to C₁₂ hydrocarbon-based groups.

109. The composition according to claim 82, wherein in said formula (I), R⁴, which can be identical or different, are each chosen from hydrogen atoms.

110. The composition according to claim 82, wherein said at least one polymer of formula (I) is in the form of a mixture of polymers, wherein said mixture optionally also comprises a compound of formula (I) wherein n is equal to zero.

111. The composition according to claim 82, wherein said at least one polyamide polymer is chosen from polymers resulting from at least one polycondensation reaction between at least one dicarboxylic acid comprising at least 32 carbon atoms and at least one amine chosen from diamines comprising at least 2 carbon atoms and triamines comprising at least 2 carbon atoms.

112. The composition according to claim 82, wherein said at least one dicarboxylic acid comprises from 32 to 44 carbon atoms and said at least one amine comprises from 2 to 36 carbon atoms.

113. The composition according to claim 112, wherein said at least one dicarboxylic acid is chosen from dimers of at least one fatty acid comprising at least 16 carbon atoms.

114. The composition according to claim 113, wherein said at least one fatty acid is chosen from oleic acid, linoleic acid and linolenic acid.

115. The composition according to claim 114, wherein said at least one amine is chosen from ethylenediamine, hexylenediamine, hexamethylenediamine, phenylenediamine and ethylenetriamine.

116. The composition according to claim 82, wherein said at least one polyamide polymer is chosen from polymers comprising at least one terminal carboxylic acid group.

117. The composition according to claim 116, wherein said at least one terminal carboxylic acid group is esterified with at least one alcohol chosen from monoalcohols comprising at least 4 carbon atoms.

118. The composition according to claim 117, wherein said at least one polyamide polymer is chosen from:
polymers chosen from mixtures of copolymers derived from monomers of (i) C₃₆ diacids and (ii) ethylenediamine, and having a weight-average molecular mass of about 6000;
polyamide polymers resulting from the condensation of at least one aliphatic dicarboxylic acid and at least one diamine, the carbonyl and amine groups being condensed via an amide bond; and
polyamide resins from vegetable sources.

119. The composition according to claim 82, wherein said at least one polyamide polymer has a softening point greater than 50° C.

120. The composition according to claim 119, wherein said at least one polyamide polymer has a softening point ranging from 65° C. to 190° C.

121. The composition according to claim 120, wherein said at least one polyamide polymer has a softening point ranging from 70° C. to 130° C.

122. The composition according to claim 121, wherein said at least one polyamide polymer has a softening point ranging from 80° C. to 105° C.

123. The composition according to claim 118, wherein said at least one polyamide polymer is present in the composition in an amount ranging from 0.5% to 80% by weight relative to the total weight of the composition.

124. The composition according to claim 123, wherein said at least one polyamide polymer is present in the composition in an amount ranging from 2% to 60% by weight relative to the total weight of the composition.

125. The composition according to claim 124, wherein said at least one polyamide polymer is present in the composition in an amount ranging from 5% to 40% by weight relative to the total weight of the composition.

126. The composition according to claim 82, wherein said at least one liquid fatty phase of the composition comprises at least one oil.

127. The composition according to claim 126, wherein said at least one oil is chosen from at least one polar oil and at least one apolar oil.

128. The composition according to claim 127, wherein said at least one polar oil is chosen from:
hydrocarbon-based plant oils with a high content of triglycerides comprising fatty acid esters of glycerol in which the fatty acids comprise chains having from 4 to 24 carbon atoms, said chains optionally being chosen from linear and branched, and saturated and unsaturated chains;
synthetic oils or esters of formula R₅COOR₆ in which R₅ is chosen from linear and branched fatty acid residues comprising from 1 to 40 carbon atoms and R₅+R₆>10;
synthetic ethers containing from 10 to 40 carbon atoms;
C₈ to C₂₆ fatty alcohols; and
C₈ to C₂₆ fatty acids.

129. The composition according to claim 127, wherein said at least one apolar oil is chosen from:
silicone oils chosen from volatile and non-volatile, linear and cyclic polydimethylsiloxanes that are liquid at room temperature;
polydimethylsiloxanes comprising alkyl or alkoxy groups which are pendant and/or at the end of the silicone chain, the groups each containing from 2 to 24 carbon atoms;
phenylsilicones; and
hydrocarbons chosen from linear and branched, volatile and non-volatile hydrocarbons of synthetic and mineral origin.

130. The composition according to claim 126, wherein said at least one liquid fatty phase comprises at least one non-volatile oil.

131. The composition according to claim 130, wherein said at least one non-volatile oil is chosen from hydrocarbon-based oils of mineral, plant and synthetic origin, synthetic esters and ethers, and silicone oils.

132. The composition according to claim 131, wherein said at least one liquid fatty phase is present in an amount ranging from 1% to 99% by weight relative to the total weight of the composition.

133. The composition according to claim 132, wherein said at least one liquid fatty phase is present in an amount ranging from 5% to 95.5% by weight relative to the total weight of the composition.

134. The composition according to claim 133, wherein said at least one liquid fatty phase is present in an amount ranging from 10% to 80% by weight relative to the total weight of the composition.

135. The composition according to claim 134, wherein said at least one liquid fatty phase is present in an amount ranging from 20% to 75% by weight relative to the total weight of the composition.

136. The composition according to claim 126, wherein said at least one liquid fatty phase comprises at least one volatile solvent chosen from hydrocarbon-based solvents and silicone solvents optionally comprising alkyl or alkoxy groups that are pendant or at the end of a silicone chain.

137. The composition according to claim 136, wherein said at least one volatile solvent is present in an amount up to 95.5% relative to the total weight of the composition.

138. The composition according to claim 137, wherein said at least one volatile solvent is present in an amount ranging from 2% to 75% relative to the total weight of the composition.

139. The composition according to claim 138, wherein said at least one volatile solvent is present in an amount ranging from 10% to 45% relative to the total weight of the composition.

140. The composition according to claim 126, wherein said composition further comprises at least one additional fatty material.

141. The composition according to claim 140, wherein said at least one additional fatty material is chosen from gums, fatty materials pasty at ambient temperature, and resins.

142. The composition according to claim 99, wherein the composition is in a form chosen from a fluid simple emulsion, rigid simple emulsion, fluid multiple emulsion, and rigid multiple emulsion.

143. The cosmetic composition of claim 82, wherein said swelling agent comprises linear or cyclic polydimethylsiloxane.

144. The cosmetic composition of claim 143, wherein said polydimethylsiloxane comprises a cyclomethicone.

145. The cosmetic composition of claim 143, wherein said polydimethylsiloxane comprises a dimethicone.

146. The cosmetic composition of claim 82, wherein said swelling agent comprises a phenylmethicone.

147. The cosmetic composition of claim 82, wherein said swelling agent comprises a fluorinated silicone.

148. The cosmetic composition of claim 82, wherein said silicone resin comprises a polyorganosilsesquioxane.

149. The cosmetic composition of claim 82, wherein said silicone elastomer core is unfunctionalized.

150. The cosmetic composition of claim 82, wherein said silicone elastomer core contains pendant functional groups.

151. The cosmetic composition of claim 150, wherein said functional groups comprise fluoroalkyl groups.

152. The cosmetic composition of claim 150, wherein said functional groups comprise phenyl groups.

153. The cosmetic composition of claim 82, wherein said structuring agent comprises a polyamide bonded to a fatty chain via an ester group, said swelling agent comprises a dimethicone, and said silicone resin comprises a polyorganosilsesquioxane.

154. The cosmetic composition of claim 82, wherein ratio of amount of said silicone elastomer powder to said structuring agent is from about 0.1 to about 9.0.

155. The cosmetic composition of claim 154, wherein the ratio is from about 0.5 to about 5.0.

156. The cosmetic composition of claim 154, wherein the ratio is from about 1.0 to about 4.0.

157. The cosmetic composition of claim 154, wherein the ration is from about 1.0 to about 3.0.

158. A method for increasing solar protection comprising the application of a composition according to claim 1.

159. A method for increasing solar protection comprising the application of a composition according to claim 99.

160. A foundation, mascara, eye liner, concealer, lipstick, blush for cheeks or eyelids, body makeup, sun screen, deodorant, colorant for skin or hair, skin care formula, shampoo, after shampoo treatment, or makeup removing product comprising:
at least one liquid fatty phase in said foundation, mascara, eye liner, concealer, lipstick, blush for cheeks or eyelids, body makeup, sun screen, deodorant, colorant for skin or hair, skin care formula, shampoo, after shampoo treatment, or makeup removing product which comprises:
(i) at least one structuring polymer comprising:
a polymer skeleton which comprises at least one hydrocarbon-based repeating unit comprising at least one hetero atom;
(ii) at least one sunscreen agent; and
(iii) a silicone elastomer powder comprising a silicone elastomer core coated with a silicone resin; and
(iv) a swelling agent for said powder.

161. A make-up and/or care and/or treatment composition for keratinous fibers comprising:
at least one liquid fatty phase in said composition which comprises:
(i) at least one structuring polymer comprising:
a polymer skeleton which comprises at least one hydrocarbon-based repeating unit comprising at least one hetero atom;
(ii) at least one sunscreen agent; and
(iii) a silicone elastomer powder comprising a silicone elastomer core coated with a silicone resin; and
(iv) a swelling agent for said powder.

162. A treatment, care or make-up composition for keratinous fibers comprising a structured composition containing at least one liquid fatty phase in said treatment, care or make-up composition structured with at least one structuring polymer comprising a polymer skeleton comprising at least one hydrocarbon-based repeating unit comprising at least one hetero atom, at least one sunscreen agent, a silicone elastomer powder comprising a silicone elastomer core coated with a silicone resin; a swelling agent for said powder, and at least one coloring agent.

163. A method for care, make up, or treatment of a keratin material chosen from lips, skin, and keratinous fibers, comprising the application to said keratin material of a cosmetic composition comprising:
at least one liquid fatty phase which comprises:
(i) at least one structuring polymer comprising:
a polymer skeleton which comprises at least one hydrocarbon-based repeating unit comprising at least one hetero atom;
(ii) at least one sunscreen agent; and
(iii) a silicone elastomer powder comprising a silicone elastomer core coated with a silicone resin; and
(iv) a swelling agent for said powder.

164. A method for making a cosmetic composition in the form of a physiologically acceptable composition, comprising including in said composition at least one liquid fatty phase which comprises:
(i) at least one structuring polymer comprising:
a polymer skeleton which comprises at least one hydrocarbon-based repeating unit comprising at least one hetero atom;
(ii) at least one sunscreen agent;
(iii) a silicone elastomer powder comprising a silicone elastomer core coated with a silicone resin; and
(iv) a swelling agent for said powder.
